(19) United States
(12) Patent Application Publication
(10) Pub. No.: US 2023/0288361 A1
(43) Pub. Date: Sep. 14, 2023

(54) CHEMIRESISTOR AND METHOD OF MANUFACTURING THE SAME AND CHEMIRESISTIVE SENSOR AND DEVICE

(71) Applicants: UNIST(ULSAN NATIONAL INSTITUTE OF SCIENCE AND TECHNOLOGY), Ulsan (KR); IUCF-HYU (Industry-University Cooperation Foundation Hanyang University), Seoul (KR); KOREA ADVANCED INSTITUTE OF SCIENCE AND TECHNOLOGY, Daejeon (KR)

(72) Inventors: Hoi Ri MOON, Ulsan (KR); Jae Hwa LEE, Ulsan (KR); Sujee CHO, Ulsan (KR); Seoyeon SEONG, Ulsan (KR); Minhyuk KIM, Ulsan (KR); Jihan KIM, Daejeon (KR); Ohmin KWON, Daejeon (KR); Mingyu JEON, Daejeon (KR); IL Doo KIM, Daejeon (KR); Chungseong PARK, Daejeon (KR); Joon-Seok LEE, Seoul (KR); Seon-Jin CHOI, Seoul (KR)

(21) Appl. No.: 18/120,277

(22) Filed: Mar. 10, 2023

(30) Foreign Application Priority Data
Mar. 11, 2022 (KR) 10-2022-0030852
Mar. 6, 2023 (KR) 10-2023-0029375

(51) Int. Cl. G01N 27/12; G01N 33/00

(52) U.S. Cl. CPC G01N 27/126; G01N 33/0027

(57) ABSTRACT

Provided are a chemiresistor and a method of manufacturing the same, a chemiresistive sensor, and a device. The chemiresistor includes a conductive porous nanocomposite of a three-dimensional metal-organic framework and a two-dimensional metal-organic framework, wherein the two-dimensional metal-organic framework is chemically bound to the three-dimensional metal-organic framework on a surface of the three-dimensional metal-organic framework, and the three-dimensional metal-organic framework and the two-dimensional metal-organic framework form a core-shell structure in the conductive porous nanocomposite.

### CROSS-REFERENCE TO RELATED APPLICATION

This application claims priorities to and the benefit of Korean Patent Applications No. 10-2022-0030852 filed in the Korean Intellectual Property Office on Mar. 11, 2022, and Korean Patent Application No. 10-2023-0029375 filed in the Korean Intellectual Property Office on Mar. 6, 2023, the entire contents of which are incorporated herein by reference.

### BACKGROUND OF THE INVENTION

### (A) Field of the Invention

A chemiresistor, a method of manufacturing the same, a chemiresistive sensor, and a device are disclosed.

### (b) Description of the Related Art

A chemiresistor is a material that changes its electrical resistance in response to changes in types and/or concentration of gases, and may be used to detect or sense gases such as toxic gases or harmful gases.

### SUMMARY OF THE INVENTION

An embodiment provides a chemiresistor with improved gas sensing performance and gas selectivity.

Another embodiment provides a method of manufacturing the chemiresistor. Yet another embodiment provides a chemiresistive sensor including the chemiresistor.

Yet another embodiment provides a device including the chemiresistor or the chemiresistive sensor.

According to an embodiment, a chemiresistor includes a conductive porous nanocomposite of a three-dimensional metal-organic framework and a two-dimensional metal-organic framework, wherein the two-dimensional metal-organic framework is chemically bound to the three-dimensional metal-organic framework on a surface of the three-dimensional metal-organic framework, and the three-dimensional metal-organic framework and the two-dimensional metal-organic framework form a core-shell structure in the conductive porous nanocomposite.

The two-dimensional metal-organic framework may include a plurality of two-dimensional hexagonal layers stacked with each other, and each of the plurality of two-dimensional hexagonal layers is derived from a combination of an organic ligand having a hydrogen bonding functional group and a metal cluster.

The organic ligand of the two-dimensional metal-organic framework may include a benzene ring or a fused polycyclic aromatic ring substituted with one or more selected from a hydroxy group, an amino group, a thiol group, or a combination thereof.

The metal cluster of the two-dimensional metal-organic framework may include Cu²⁺, Ni²⁺, Co²⁺, Fe²⁺, Zn²⁺, Mg²⁺, or combination thereof.

Each of the plurality of two-dimensional hexagonal layers of the two-dimensional metal-organic framework may be vertically aligned to the surface of the three-dimensional metal-organic framework.

The plurality of two-dimensional hexagonal layers may include a first two-dimensional hexagonal layer and a second two-dimensional hexagonal layer that are alternatively stacked, and a coordination number of the metal cluster of the second two-dimensional hexagonal layer may be the same as or different from a coordination number of the metal cluster of the first two-dimensional hexagonal layer.

The metal cluster included in the first two-dimensional hexagonal layer and the metal cluster included in the second two-dimensional hexagonal layer may be arranged side by side or zigzag along a direction perpendicular to an in-plane direction of the first and second two-dimensional hexagonal layers.

The organic ligand of the two-dimensional metal-organic framework may be coordinated with a metal cluster of the three-dimensional metal-organic framework at the interface of the three-dimensional metal-organic framework and the two-dimensional metal-organic framework.

The coordination number of the metal cluster of the two-dimensional metal-organic framework at the interface of the three-dimensional metal-organic framework and the two-dimensional metal-organic framework may be higher than the coordination number of the metal cluster of the two-dimensional metal-organic framework in a region other than the interface of the three-dimensional metal-organic framework and the two-dimensional metal-organic framework.

The three-dimensional metal-organic framework may be an octahedral porous material, and the two-dimensional metal-organic framework may be a rod-shaped conductive material.

According to another embodiment, a method of manufacturing a chemiresistor includes surface-modifying a three-dimensional metal-organic framework with an organic ligand having a hydrogen bonding functional group for a two-dimensional metal-organic framework, and providing a metal precursor for the two-dimensional metal-organic framework to the surface-modified three-dimensional metal-organic framework and then performing seed-mediated crystal growth to form a conductive porous nanocomposite of the three-dimensional metal-organic framework and the two-dimensional metal-organic framework with a core-shell structure, the two-dimensional metal-organic framework being chemically bound to the three-dimensional metal-organic framework on a surface of the three-dimensional metal-organic framework.

The surface-modifying may include preparing a mixed dispersion including the three-dimensional metal-organic framework and the organic ligand for the two-dimensional metal-organic framework, and ultrasonicating the mixed dispersion.

The mixed dispersion may further include N,N-diethyl formamide.

The organic ligand for a two-dimensional metal-organic framework may include a benzene ring or a fused polycyclic aromatic ring substituted with one or more hydrogen bonding functional groups selected from a hydroxy group, an amino group, a thiol group, or a combination thereof.

The benzene ring or the fused polycyclic aromatic ring may have six hydrogen bonding functional groups.

The metal cluster for the two-dimensional metal-organic framework may include a metal cation and a counter anion, and the metal cation may include Cu²⁺, Ni²⁺, Co²⁺, Fe²⁺, Zn²⁺, Mg²⁺, or a combination thereof.

The seed-mediated crystal growth may include heat-treating the mixture of the surface-modified three-dimensional metal-organic framework and the metal precursor for the two-dimensional metal-organic framework at a temperature of about 40° C. to about 100° C.

According to another embodiment, a chemiresistive sensor including the chemiresistor is provided.

The chemiresistive sensor may be a gas sensor for detecting hydrogen sulfide gas.

According to another embodiment, a device including the chemiresistor or the chemiresistive sensor is provided.

Gas detection performance and gas selectivity at room temperature may be improved.

### BRIEF DESCRIPTION OF THE DRAWINGS

FIG. 1 is a schematic view of a chemiresistor according to an embodiment,

FIG. 2A is a schematic view showing an example of a core-shell structure of the chemiresistor of FIG. 1,

FIG. 2B is a schematic view showing an enlarged interface of the three-dimensional metal-organic framework and the two-dimensional metal-organic framework of the chemiresistor of FIG. 2A,

FIG. 3 is a schematic view showing an example of a method for manufacturing a chemiresistor according to an embodiment,

FIG. 4 is a schematic view showing an example of a chemiresistive sensor according to an embodiment,

FIG. 5A is a SEM image of the Ni—HHTP@UiO-66-NH₂ composites obtained in Preparation Example,

FIG. 5B is a SEM image of the UiO-66-NH₂ core crystals obtained in Reference Preparation Example 1,

FIG. 5C is a SEM image of the Ni—HHTP rod crystals obtained in Reference Preparation Example 2,

FIG. 6 is a TEM image of the Ni—HHTP@UiO-66-NH₂ composites obtained in Preparation Example,

FIGS. 7A and 8A are TEM images of lattice fringe analysis of the Ni—HHTP rod crystals of the Ni—HHTP@UiO-66-NH₂ composites obtained in Preparation Example,

FIGS. 7B and 8B are schematic views showing the lattice fringe analysis results of FIGS. 7A and 8A, respectively,

FIGS. 9, 10A, and 10B are electron microscopy images of the interface of the UiO-66-NH₂ core crystal and the Ni—HHTP rod crystal of the Ni—HHTP@UiO-66-NH₂ composites obtained in Preparation Example,

FIG. 10C is a schematic view showing microscopic image of the interface of the UiO-66-NH₂ core crystal and the Ni—HHTP rod crystal of FIG. 10B,

FIG. 11 is an electron paramagnetic resonance (EPR) graph of the Ni—HHTP@UiO-66-NH₂ composites obtained in Preparation Example, UiO-66-NH₂ crystals obtained in Reference Preparation Example 1, and Ni—HHTP rod crystals obtained in Reference Preparation Example 2,

FIGS. 12 and 13 are X-ray absorption fine structure (XAFS) graphs of the Ni—HHTP@UiO-66-NH₂ composites obtained in Preparation Example,

FIG. 14 is a graph showing changes in sensitivity when the chemiresistive sensors according to Example and Reference Example are exposed to hydrogen sulfide gas H₂S,

FIG. 15 is a graph showing gas sensitivity according to gas concentration of the chemiresistive sensor according to Example,

FIG. 16 is a graph showing a relationship between gas concentration and change in sensitivity of the chemiresistive sensor according to Example, and

FIG. 17 is a graph showing gas selectivity of the chemiresistive sensors according to Example and Reference Example.

### DETAILED DESCRIPTION OF THE EMBODIMENTS

Hereinafter, embodiments of the present disclosure will be described in detail so that a person skilled in the art would understand the same. This disclosure may, however, be embodied in many different forms and is not construed as limited to the example embodiments set forth herein.

In the drawings, the thickness of layers, films, panels, regions, etc., are exaggerated for clarity. Like reference numerals designate like elements throughout the specification. It will be understood that when an element such as a layer, film, region, or substrate is referred to as being “on” another element, it can be directly on the other element or intervening elements may also be present. In contrast, when an element is referred to as being “directly on” another element, there are no intervening elements present.

Hereinafter, the term “combination” includes a mixture and a stacked structure of two or more.

Hereinafter, the term “metal” includes metal and semi-metal.

As used herein, when specific definition is not otherwise provided, “substituted” refers to replacement of a hydrogen of a compound by a substituent of a halogen atom, a hydroxy group, an alkoxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid group or a salt thereof, a silyl group, a C1 to C20 alkyl group, a C2 to C20 alkenyl group, a C2 to C20 alkynyl group, a C6 to C30 aryl group, a C7 to C30 arylalkyl group, a C1 to C30 alkoxy group, a C1 to C20 heteroalkyl group, a C3 to C20 heteroaryl group, a C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, or a combination thereof.

Hereinafter, a chemiresistor according to an embodiment is described.

A chemiresistor is a material configured to show a change in electrical resistance in response to a change in the surrounding chemical environment, and may show a change in electrical resistance according to a chemical interaction between the chemiresistor and surrounding gases.

A chemiresistor according to an embodiment includes metal-organic frameworks (MOFs) based material formed by self-assembly of organic ligands (or organic linkers) and metal clusters (or metal nodes), and may include nanocomposites of a plurality of metal-organic frameworks that are structurally and functionally different from each other.

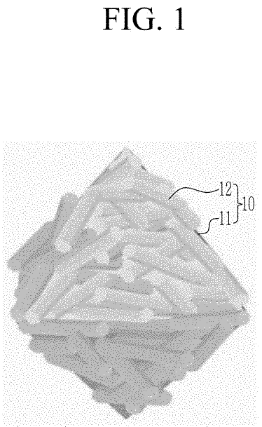

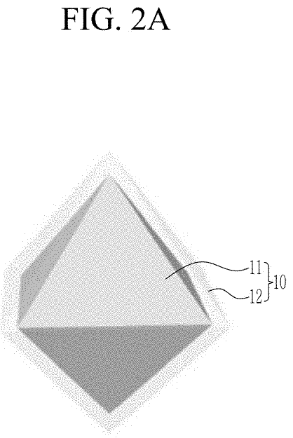

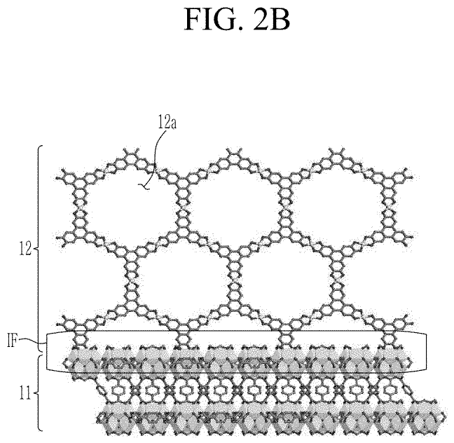

FIG. 1 is a schematic view of a chemiresistor according to an embodiment, FIG. 2A is a schematic view showing an example of a core-shell structure of the chemiresistor of FIG. 1, and FIG. 2B is a schematic view showing an enlarged interface of the three-dimensional metal-organic framework and the two-dimensional metal-organic framework of the chemiresistor of FIG. 2A.

Referring to FIGS. 1, 2A, and 2B, the chemiresistor according to an embodiment may be or include a nanocomposite of a three-dimensional metal-organic framework (3D-MOF) 11 and a two-dimensional metal-organic framework (2D-MOF) 12 that are structurally and functionally different from each other, and it may be a conductive porous nanocomposite.

The three-dimensional metal-organic framework 11 and the two-dimensional metal-organic framework 12 may form a core-shell structure in the conductive porous nanocomposite, and the two-dimensional metal-organic framework 12 may cover a part or all of the surface of a three-dimensional metal-organic framework 11.

The three-dimensional metal-organic framework 11 may be a porous metal-organic framework having a three-dimensional structure formed by self-assembly of organic ligands and metal clusters, wherein the three-dimensional structure may have a polyhedral shape with spaces in X, Y, and Z directions. Due to such a three-dimensional structure, the three-dimensional metal-organic framework 11 may have a high porosity and surface area, and thus it may exhibit high adsorption and capacity for gases such as gas molecules.

FIGS. 1 and 2A show an octahedron structure as an example, however it is not limited thereto, and the three-dimensional metal-organic framework 11 may have, for example, a tetrahedron structure, a cube structure, a dodecahedron structure, an icosahedron structure, or a combination thereof.

The organic ligands and metal clusters of the three-dimensional metal-organic framework 11 may be selected in consideration of a pair capable of forming the above-described porous three-dimensional structure, high chemical stability, hydrothermal stability, and gas-accepting capacity.

The organic ligands for the three-dimensional metal-organic framework 11 may be, for example, a non-polymer or a non-conductive monomer.

For example, the organic ligands for the three-dimensional metal-organic framework 11 may include an aromatic compound having one or more carboxylic groups, and for example, 2-amino-1,4-benzene dicarboxylate (BDC), 2,5-dioxodo-1,4-benzenedicarboxylate (DOBDC), 1,3,5-benzenetricarboxylate (H₂BTC), or a combination thereof, but are not limited thereto.

The metal cluster for the three-dimensional metal-organic framework 11 may include Zr²⁺, Cu²⁺, Ni²⁺, Co²⁺, Fe²⁺, Zn²⁺, Mg²⁺, Cr²⁺, or a combination thereof, but is not limited thereto.

The three-dimensional metal-organic framework 11 may include for example UiO-66-NH₂, M-MOF-74, M-MIL-101, M-MOF-808 (wherein M is Zr, Cu, Ni, Co, Fe, Zn, Mg, Cr, or a combination thereof), or a combination thereof, but is not limited thereto. The three-dimensional metal-organic framework 11 may form a core of a core-shell structure of the conductive porous nanocomposite.

The two-dimensional metal-organic framework 12 may be a conductive metal-organic framework having a two-dimensional structure formed by self-assembly of organic ligands and metal clusters, wherein the two-dimensional structure may be a planar structure extending in an in-plane direction (e.g., XY direction) or a layered structure in which a plurality of planar structures are stacked.

For example, the two-dimensional metal-organic framework 12 may have a long rod shape in one direction including a layered structure in which a plurality of two-dimensional hexagonal layers to be described later are stacked, wherein a length direction of the long rod shape may be the same as the stacked direction of the layered structure.

The two-dimensional metal-organic framework 12 may provide high conductivity to the chemiresistor 10 by forming high conjugation along the in-plane direction of the layered structure and/or between adjacent layered structures.

The organic ligands for the two-dimensional metal-organic framework 12 may be different from the organic ligands for the three-dimensional metal-organic framework 11, and the metal clusters for the two-dimensional metal-organic framework 12 may be the same as or different from the metal clusters for the three-dimensional metal-organic framework 11.

The organic ligands and metal clusters for the two-dimensional metal-organic framework 12 may be selected in the light of conductivity and a pair that can provide high binding energy with target gases (e.g., gas molecules such as hydrogen sulfide, carbon monoxide, or combinations thereof).

The organic ligands for the two-dimensional metal-organic framework 12 may be organic ligands capable of forming a hydrogen bonding, for example, it may include a benzene ring or a fused polycyclic aromatic ring substituted with one or more hydrogen bonding functional groups selected from a hydroxyl group (—OH), an amino group (—NH₂), a thiol group (SH), or a combination thereof.

The fused polycyclic aromatic ring may include two to ten of hydrocarbon aromatic rings, heteroaromatic rings, or a combination thereof, fused with each other.

The organic ligands capable of forming hydrogen bonding may be represented by, for example, any one of Chemical Formulas 1 to 4, but are not limited thereto.

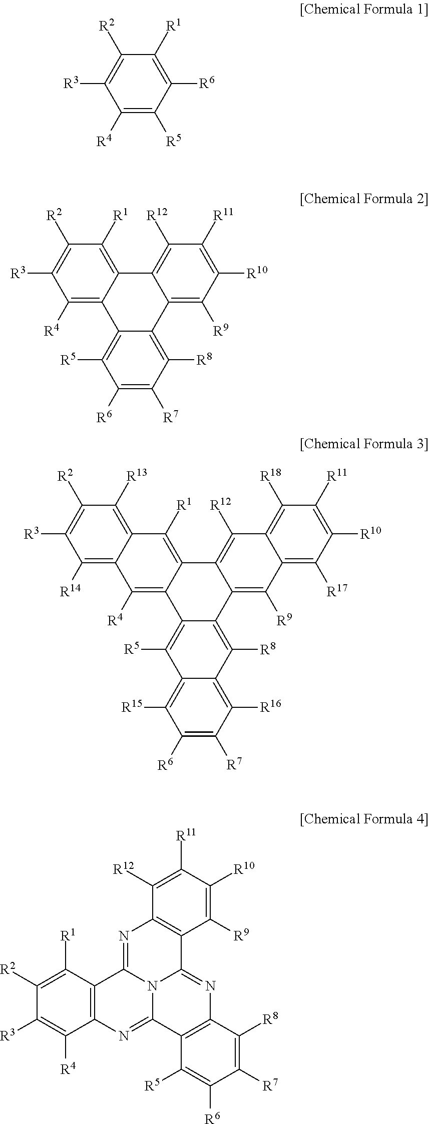

In Chemical Formula 1 to 4,
R¹ to R¹⁸ are each independently hydrogen, a substituted or unsubstituted C1 to C30 alkyl group, a substituted or unsubstituted C1 to C30 alkoxy group, a substituted or unsubstituted C6 to C20 aryl group, a substituted or unsubstituted C3 to C20 heterocyclic group, a halogen, a cyano group, or combination thereof, and
at least one of R¹ to R¹⁸ are a hydrogen bonding functional group, for example, a hydroxyl group (—OH), an amino group (—NH₂), a thiol group (SH), or a combination thereof.

As an example, the organic ligands may have six hydrogen bonding functional groups, thereby forming a two-dimensional hexagonal layer described later.

For example, R¹ to R⁶ in Chemical Formula 1 may each be a hydrogen bonding functional group, and for example, each of R², R³, R⁶, R⁷, R¹⁰, and R¹¹ in Chemical Formulas 2 to 4 may be a hydrogen bonding functional group.

Six hydrogen bonding functional groups may be reactive sites capable of coordinating with the metal clusters.

For example, the organic ligands may include hexahydroxybenzene, hexaaminebenzene, hexathiolbenzene, hexahydroxytriphenylene, hexaaminetriphenylene, hexathiol triphenylene, hexahydroxytrinaphthylene, hexaaminetrinaphthylene, hexathioltrinaphthylene, or a combination thereof, for example, triphenylene-2,3,6,7,10,11-hexaamine, triphenylene-2,3,6,7,10,11-hexol, triphenylene-2,3,6,7,10,11-hexathiol, Trinaphthylene-3,4,9,10,15,16-hexaamine, trinaphthylene-3,4,9,10,15,16-hexol, trinaphthylene-3,4,9,10,15,16-hexathiol, or a combination thereof, but are not limited thereto.

The metal clusters of the two-dimensional metal-organic framework 12 may include, for example, Cu²⁺, Ni²⁺, Co²⁺, Fe²⁺, Zn²⁺, Mg²⁺, or a combination thereof, but is not limited thereto. The two-dimensional metal-organic framework 12 may form a shell of a core-shell structure of the conductive porous nanocomposite.

The two-dimensional metal-organic framework 12 may include a plurality of two-dimensional hexagonal layers derived from self-assembly of the organic ligands and the metal clusters.

Each two-dimensional hexagonal layer may have a honeycomb shape with a plurality of hexagonal pores 12a, and complexes of the organic ligands and the metal clusters continuously arranged may be around the hexagonal pores 12a.

The continuous arrangement of the complexes of the organic ligands and the metal clusters may provide high conductivity to the chemiresistor 10. Each of the two-dimensional hexagonal layers may be substantially vertically aligned to the surface of the three-dimensional metal-organic framework 11.

For example, the two-dimensional hexagonal layer of the two-dimensional metal-organic framework 12 may be aligned at an angle of about 60 degrees to 120 degrees with respect to the surface of the three-dimensional metal-organic framework 11, and within the above range, from about 70 degrees to about 110 degrees, from about 75 degrees to about 105 degrees, from about 80 degrees to about 100 degrees, from about 85 degrees to about 95 degrees, or about 90 degrees.

A plurality of two-dimensional hexagonal layers may be stacked with each other and each of the two-dimensional hexagonal layers may be vertically aligned to the surface of the three-dimensional metal-organic framework 11.

For example, the plurality of two-dimensional hexagonal layers may include a first two-dimensional hexagonal layer and a second two-dimensional hexagonal layer that are alternately stacked, and a coordination number of the metal cluster of the second two-dimensional hexagonal layer may be the same as or different from a coordination number of the metal clusters of the first two-dimensional hexagonal layer.

For example, the metal clusters included in the first two-dimensional hexagonal layer and the metal clusters included in the second two-dimensional hexagonal layer are arranged side by side (so-called, eclipsed parallel structure) or in a zigzag pattern (so-called slipped-parallel structure) with each other along a direction perpendicular to the in-plane direction of the first and second two-dimensional hexagonal layers.

For example, the metal clusters included in the first two-dimensional hexagonal layer and the metal clusters included in the second two-dimensional hexagonal layer may be arranged in a zigzag pattern along a direction perpendicular to the in-plane direction of the first and second two-dimensional hexagonal layers, and thus due to high binding energy, interaction with gases (e.g., gas molecules such as hydrogen sulfide, carbon monoxide, or a combination thereof) may be increased, thereby increasing the binding ability with the gases.

The two-dimensional metal-organic framework 12 may be chemically bound to the three-dimensional metal-organic framework 11 on the surface of the three-dimensional metal-organic framework 11.

That is, in the core-shell structure, an interface IF of the core, which is the three-dimensional metal-organic framework 11, and the shell, which is the two-dimensional metal-organic framework 12, may include chemical bonds between the core and the shell, and the chemical bonds may be, for example, coordination bonds, covalent bonds, or a combination thereof, but is not limited thereto.

Such chemical bonds may be effectively formed by a seed-mediated reaction between the three-dimensional metal-organic framework 11 and the two-dimensional metal-organic framework 12.

Here, the chemical bonds may be substantial bonds between atoms or ions and may not include an interaction such as a Van der Waals interaction.

The interface IF of the three-dimensional metal-organic framework 11 and the two-dimensional metal-organic framework 12 may include the chemical bonds between the three-dimensional metal-organic framework 11 and the two-dimensional metal-organic framework 12, and thus the chemiresistor 10 may be a composite (nanocomposite) including chemical bonds between the three-dimensional metal-organic framework 11 and the two-dimensional metal-organic framework 12, and not a simple physical mixture of the three-dimensional metal-organic framework 11 and the two-dimensional metal-organic framework 12.

For example, the interface IF of the three-dimensional metal-organic framework 11 and the two-dimensional metal-organic framework 12 may have coordination bonds between the organic ligands capable of forming hydrogen bonding of the two-dimensional metal-organic framework 12 and the metal clusters of the three-dimensional metal-organic framework 11.

For example, the metal clusters of the two-dimensional metal-organic framework 12 at the interface IF of the three-dimensional metal-organic framework 11 and the two-dimensional metal-organic framework 12 may be different from the metal clusters of the two-dimensional metal-organic framework 12 in a region other than the interface IF of the three-dimensional metal-organic framework 11 and the two-dimensional metal-organic framework 12.

As will be described later, the two-dimensional metal-organic framework 12 may be grown in a shell form on the surface of the three-dimensional metal-organic framework 11, and during this shell growth reaction, the unmatched lattice parameters and connection points between the two-dimensional metal-organic framework 12 and the three-dimensional metal-organic framework 11 may induce structural transformation at the interface of the three-dimensional metal-organic framework 11 and the two-dimensional metal-organic framework 12.

Due to this structural transformation, a coordination number of the metal clusters of the two-dimensional metal-organic framework 12 at the interface of the three-dimensional metal-organic framework 11 and the two-dimensional metal-organic framework 12 may be higher than a coordination number of the metal clusters of the two-dimensional metal-organic framework 12 in a region other than the interface of the three-dimensional metal-organic framework 11 and the two-dimensional metal-organic framework 12.

The two-dimensional metal-organic framework 12 may be further bound at the interface IF of the three-dimensional metal-organic framework 11 and the two-dimensional metal-organic framework 12 by such a structural transformation of the two-dimensional metal-organic framework 12, and thus the two-dimensional metal-organic framework 12 may be firmly fixed to the surface of the three-dimensional metal-organic framework 11 and exhibit synergistic properties at the interface of the three-dimensional metal-organic framework 11 and the two-dimensional metal-organic framework 12.

Due to such a unique interfacial property of the three-dimensional metal-organic framework 11 and the two-dimensional metal-organic framework 12, the chemiresistor 10 may form a strongly-connected and highly homogeneous core-shell structure, and therefore a continuous conductive pathway may be effectively provided.

Accordingly, compared to the three-dimensional metal-organic framework 11 alone and/or the two-dimensional metal-organic framework 12 alone, the chemiresistor 10 may exhibit high selectivity and sensitivity for gases (e.g., gas molecules such as hydrogen sulfide, carbon monoxide, or combinations thereof) and accordingly, a synergistic effect capable of effectively detecting a target gas and effectively converting the detected gas into an electrical signal may be exhibited.

Hereinafter, a manufacturing method according to an embodiment of the above-described chemiresistor 10 will be described.

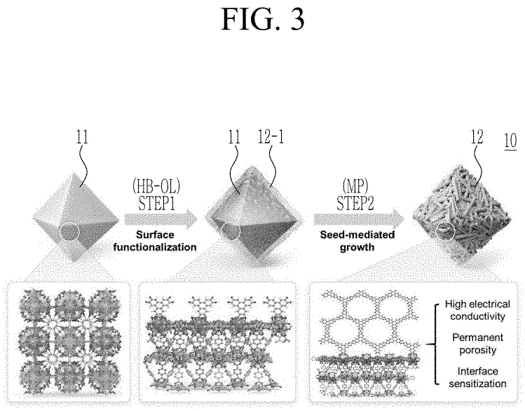

FIG. 3 is a schematic view showing an example of a method for manufacturing a chemiresistor according to an embodiment.

Referring to FIG. 3, the manufacturing method of the chemiresistor 10 according to an embodiment includes surface-modifying the three-dimensional metal-organic framework 11 with an organic ligand HB-OL having a hydrogen bonding functional group for the two-dimensional metal-organic framework to form a shell precursor 12-1 (STEP 1), and providing a metal precursor MP for the two-dimensional metal-organic framework 12 to the surface-modified three-dimensional metal-organic framework 11 and then performing seed-mediated crystal growth (STEP 2) to form a conductive porous nanocomposite of the three-dimensional metal-organic framework and the two-dimensional metal-organic framework with a core-shell structure. In the core-shell structure, the two-dimensional metal-organic framework 12 may be chemically bound to the three-dimensional metal-organic framework 11 on a surface of the three-dimensional metal-organic framework 11.

As described above, the three-dimensional metal-organic framework 11 may be a porous metal-organic framework, and it may have a three-dimensional structure of tetrahedron, hexahedron, octahedron, dodecahedron, icosahedron, or a combination thereof.

The three-dimensional metal-organic framework 11 may be, for example, UiO-66-NH₂, M-MOF-74, M-MIL-101, M-MOF-808 (wherein M may be Zr, Cu, Ni, Co, Fe, Zn, Mg, Cr, or a combination thereof), or a combination thereof, but is not limited thereto.

As described above, the organic ligand may include a benzene ring or a fused polycyclic aromatic ring substituted with one or more hydrogen bonding functional groups selected from a hydroxyl group (—OH), an amino group (—NH₂), a thiol group (—SH), or a combination thereof, and for example, it may be represented by any one of the above-described Chemical Formulas 1 to 4, and for example, the benzene ring or the fused polycyclic aromatic ring may have six hydrogen bonding functional groups.

The surface-modifying the three-dimensional metal-organic framework 11 with the organic ligand HB-OL for the two-dimensional metal-organic framework 12 may include, for example, preparing a mixed dispersion containing the three-dimensional metal-organic framework 11 and the organic ligand for the two-dimensional metal-organic framework 12 and ultrasonicating the mixed dispersion.

The mixed dispersion may be obtained by dispersing the three-dimensional metal-organic framework 11 and the organic ligand HB-OL for the two-dimensional metal-organic framework 12 in a dispersion medium together, or the three-dimensional metal-organic framework 11 and the organic ligand HB-OL for the two-dimensional metal-organic framework 12 in each dispersion medium and then mixing them.

The mixed dispersion may further include an additive, and the additive may include, for example, N,N-diethyl formamide.

N,N-diethyl formamide may act as a Lewis base in the mixed dispersion due to its high donor number and promote the seed-mediated crystal growth described later, so that the two-dimensional metal-organic framework 12 may be homogeneously attached to the surface of the three-dimensional metal-organic framework 11.

The ultrasonicating mixed dispersion may be performed for about 5 minutes to about 1 hour, and therefore the organic ligand HB-OL for the two-dimensional metal-organic framework 12 may be effectively attached and bound to the surface of the three-dimensional metal-organic framework 11.

The metal precursor MP for the two-dimensional metal-organic framework 12 may include, for example, a metal cation and a counter anion, and the metal cation may include Cu²⁺, Ni²⁺, Co²⁺, Fe²⁺, Zn²⁺, Mg²⁺, or a combination thereof.

The seed-mediated crystal growth may include heat-treating the mixture of the surface-modified three-dimensional metal-organic framework and the metal precursor for the two-dimensional metal-organic framework, and the heat-treating may be performed, for example, at a temperature of about 40° C. to about 100° C. for about 5 minutes to about 10 hours, and within the above range, from about 40° C. to about 80° C., from about 40° C. to about 70° C., or from about 40° C. to about 60° C.

Seed-mediated crystal growth may introduce the organic ligand seed capable of effectively growing the two-dimensional metal-organic framework 12 to the surface of the three-dimensional metal-organic framework 11 through the surface-modifying process of the three-dimensional metal-organic framework 11, and control the growth rate and nucleus formation rate of the two-dimensional metal-organic framework 12 through the heat-treating at a predetermined temperature.

Accordingly, it is possible to effectively prevent and/or reduce the formation of unnecessary by-products and/or abnormal two-dimensional metal-organic frameworks that do not normally grow on the surface of the three-dimensional metal-organic frameworks 11 due to excessive rapid formation during the reaction process.

As described above, the chemiresistor 10 may be formed by first surface-modifying the three-dimensional metal-organic framework 11 with the organic ligand HB-OL for the two-dimensional metal-organic framework 12, and then performing the seed-mediated crystal growth of the metal precursor MP for the two-dimensional metal-organic framework 12, and thus the two-dimensional metal-organic framework 12 may be effectively bound to the surface of the three-dimensional metal-organic framework 11. Accordingly, the two-dimensional metal-organic framework 12 may be firmly fixed to the surface of the three-dimensional metal-organic framework 11 at the interface IF of the two-dimensional metal-organic framework 12 and the three-dimensional metal-organic framework 11, and ultimately the chemiresistor 10 may exhibit a synergistic effect capable of effectively detecting a target gas and converting the detected gas into an electrical signal.

The aforementioned chemoresistor 10 may be included in a chemiresistive sensor as an active material.

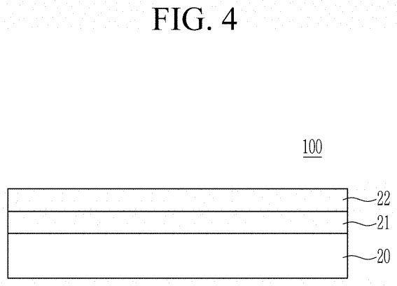

FIG. 4 is a schematic view showing an example of a chemiresistive sensor according to an embodiment.

Referring to FIG. 4, a chemiresistive sensor according to an embodiment includes a substrate 20, an electrode 21, and a chemiresistive layer 22.

The substrate 20 may be made of an inorganic material such as glass; organic materials such as polycarbonate, poly(methyl)methacrylate, polyethylene terephthalate, polyethylene naphthalate, polyimide, polyamide, polyamideimide, polyethersulfone, or combinations thereof; or silicon, but is not limited thereto.

The electrode 21 may include a conductor, for example, a metal such as Al, Ag, Au, Ni, or an alloy thereof; a conductive metal oxide such as zinc oxide, indium oxide, tin oxide, indium tin oxide ITO, indium zinc oxide IZO or fluorine doped tin oxide; nanostructures such as silver nanostructures and carbon nanostructures; or a combination thereof, but is not limited thereto.

The chemiresistive layer 22 may be an active layer and may include the chemiresistor 10 described above.

The chemiresistive layer 22 may be formed by coating a dispersion containing the chemiresistor 10 described above and drying it.

The chemiresistive layer 22 may exhibit high selectivity and sensitivity to gas molecules such as hydrogen sulfide, carbon monoxide, or a combination thereof at ambient temperature (at room temperature, about 25° C.) as well as high conductivity, thereby effectively detecting the target gas and converting into electrical signals.

The chemiresistive sensor 100 may be applied to various devices and may be included in a sensor device, a control device, an air purifier, a display device, or a wireless device capable of measuring and monitoring gases such as gas molecules in real time, but is not limited thereto.

Hereinafter, the embodiments are illustrated in more detail with reference to examples. However, these are only examples, and the scope of claims is not limited thereto.

### Synthesis of Core-Shell Structured Chemiresistor

### Reference Preparation Example 1

290 mg of 2-amino-1,4-benzenedicarboxylic acid (Sigma-Aldrich) is dissolved in 100 mL of N,N-dimethylformamide to prepare a first solution. Separately, 516 mg of zirconium (IV) chloride octahydrate (Sigma-Aldrich) and 27 mL of acetic acid are dissolved in 100 mL of N,N-dimethylformamide to prepare a second solution. Subsequently, after mixing the first solution and the second solution, the mixture is heated at 120° C. for 24 hours, and centrifuged to obtain powders. Then, the powders are washed 3 times with methanol and 3 times with acetone, and then dried in vacuum at 80° C. for 18 hours to obtain UiO-66-NH₂ (C₄₈H₃₄N₆O₃₂Zr₆) crystals (UiO-66-NH₂ core crystals).

### Preparation Example 1

15 mg of UiO-66-NH₂ crystals obtained in Reference Preparation Example 1 are added to the solution of deionized water (2 mL) and 1-propanol (2 mL) with 2,3,6,7,10,11-hexahydroxytriphenylene hydrate (3.5 mg, 0.011 mmol, HHTP, TCI Japan), and then ultrasonicated using CPX388H Ultrasonic Cleaner (Branson, Bransonic®) with a frequency of 40 kHz for 10 minutes in a vial (20 mL capacity) at room temperature. Then, 500 μL of N,N-diethylformamide (DEF) are added to the solution to prepare an HHTP@UiO-66-NH₂ dispersion. Then, nickel(II) acetate tetrahydrate (3 mg, 0.012 mmol, Ni(CH₃CO₂)₂·4H₂O) in 2 mL of deionized water and 2 mL of 1-propanol is dissolved and added to the HHTP@UiO-66-NH₂ dispersion to prepare a Ni—HHTP@UiO-66-NH₂ precursors. The Ni—HHTP@UiO-66-NH₂ precursors are heated at 55° C. for 3 hours and then centrifuged to obtain powders. The powders are washed 3 times with deionized water and 3 times with acetone, and finally dried in vacuum at 60° C. for 3 hours to obtain Ni—HHTP@UiO-66-NH₂ composites (core-shell structured chemiresistor).

### Reference Preparation Example 2

210 mg of 2,3,6,7,10,11-hexahydroxytriphenylene hydrate (HHTP, TCI Japan) is added to a mixed solvent of 60 mL of deionized water and 60 mL of 1-propanol, and additionally 30 mg of nickel acetate tetrahydrate is added thereto to prepare a dispersion. Then, the dispersion is ultrasonicated using CPX388H Ultrasonic Cleaner (Branson, Bransonic®) with a frequency of 40 kHz for 10 minutes in a vial (20 mL capacity) at room temperature, heated at 55° C. for 3 hours, and then centrifuged to obtain powders. The powders are washed 3 times with deionized water and 3 times with acetone, and finally dried in vacuum at 60° C. for 18 hours to obtain Ni—HHTP (C₅₄H₅₄O₃₆Ni₆) rod crystals.

### Reference Preparation Example 3

Ni—HHTP rod crystals obtained in Reference Preparation Example 2 and UiO-66-NH₂ crystals obtained in Reference Preparation Example 1 are mixed in a weight ratio of 2.8:7.2 to prepare a physical mixture of Ni—HHTP rod crystals and UiO-66-NH₂ crystals.

### Evaluation I

The structure of the Ni—HHTP@UiO-66-NH₂ composites obtained in Preparation Example is evaluated.

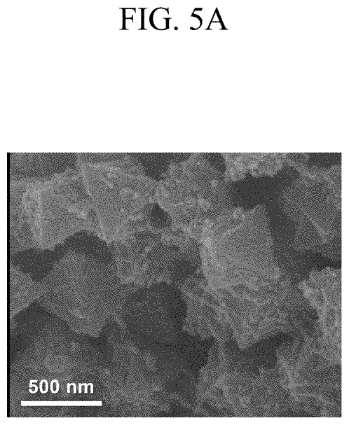

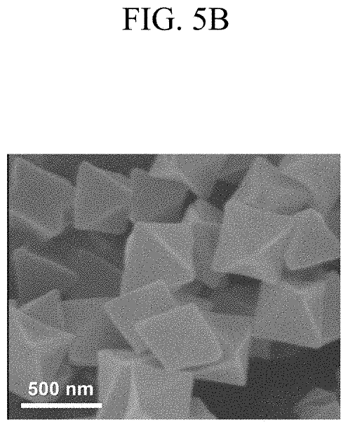

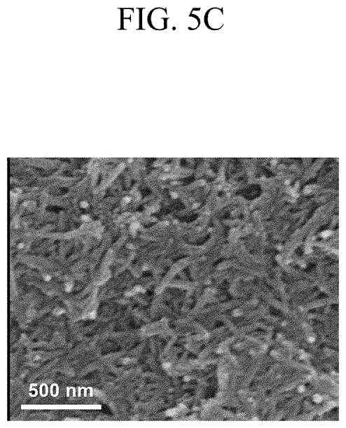

FIG. 5A is a SEM image of the Ni—HHTP@UiO-66-NH₂ composites obtained in Preparation Example, FIG. 5B is a SEM image of the UiO-66-NH₂ crystals obtained in Reference Preparation Example 1, and FIG. 5C is a SEM image of the Ni—HHTP rod crystals obtained in Reference Preparation Example 2.

Referring to FIGS. 5A to 5C, it is confirmed that the Ni—HHTP@UiO-66-NH₂ composites obtained in Preparation Example has a core-shell structure in which the octahedral UiO-66-NH₂ crystals are well-covered with the Ni—HHTP rod crystals with the length of approximately 50-100 nm, while each MOF retained their typical morphologies.

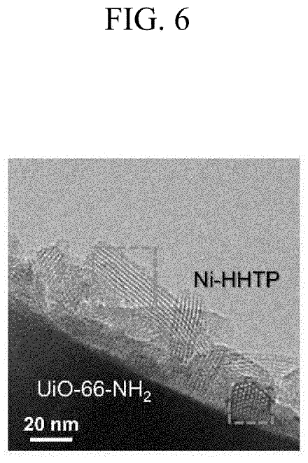

FIG. 6 is a TEM image of the Ni—HHTP@UiO-66-NH₂ composites obtained in Preparation Example.

Referring to FIG. 6, it is confirmed that the long side of Ni—HHTP rod crystals in the Ni—HHTP@UiO-66-NH₂ composites obtained in Preparation Example tends to orient parallel to the (111) facet of the UiO-66-NH₂ crystal.

In addition, X-ray spectroscopy mapping (EDS mapping) around the Ni—HHTP@UiO-66-NH₂ composites obtained in Preparation Example demonstrates that the Ni—HHTP rod crystals closely adhere to the UiO-66-NH₂ core crystals and are well-distributed throughout the octahedron UiO-66-NH₂ core crystals.

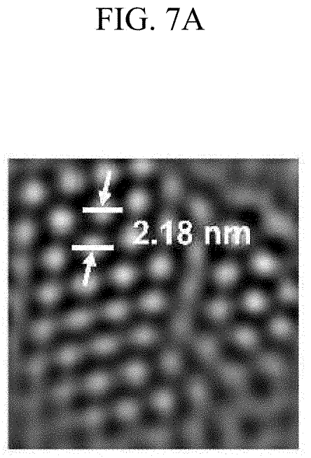

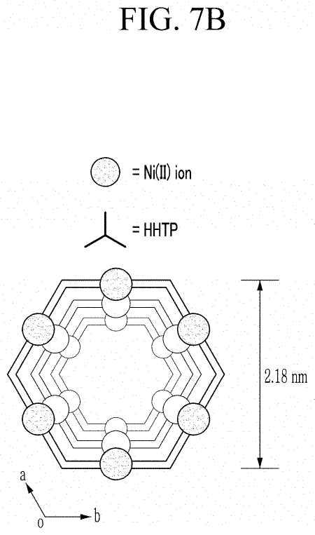

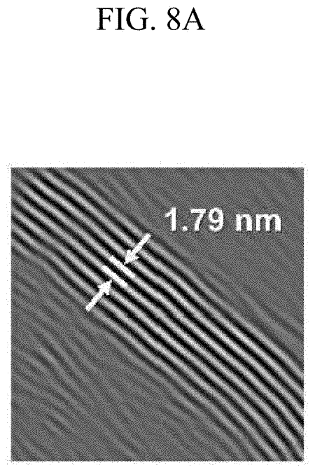

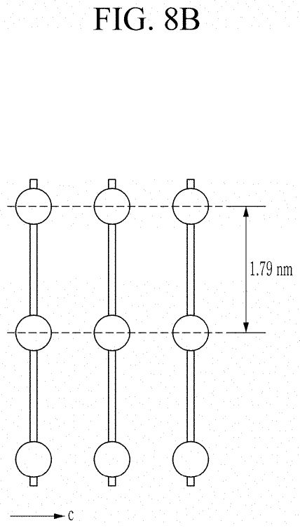

FIGS. 7A and 8A are TEM images of lattice fringe analysis of the Ni—HHTP structure of the Ni—HHTP@UiO-66-NH₂ composites obtained in Preparation Example, and FIGS. 7B and 8B are schematic views showing the lattice fringe analysis results of FIGS. 7A and 8A, respectively.

Referring to FIGS. 7A, 7B, 8A, and 8B, it is confirmed that the Ni—HHTP rod crystals in the Ni—HHTP@UiO-66-NH₂ composites obtained in Preparation Example include a plurality of layered two-dimensional hexagonal layers that are stacked with each other. Further, the lattice fringe analysis in the TEM images may confirm the formation of the Ni—HHTP rod crystals with the d-spacing values of 2.18 nm and 1.79 nm, which correspond to a center-to-center distance of the adjacent hexagonal pores and node-to-node distance along an axis, respectively.

From this, it is confirmed that two-dimensional hexagonal layers in the Ni—HHTP rod crystals are vertically aligned to the surface of the UiO-66-NH₂ core crystal.

### Evaluation II

The interface between the UiO-66-NH₂ core crystal and the Ni—HHTP rod crystal of Ni—HHTP@UiO-66-NH₂ composites obtained in Preparation Example is evaluated.

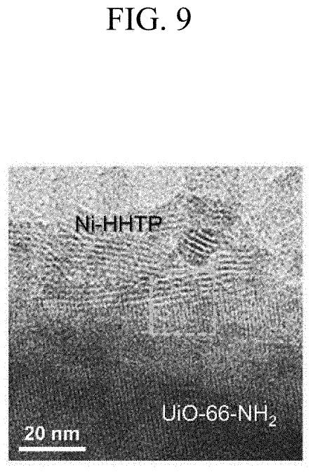

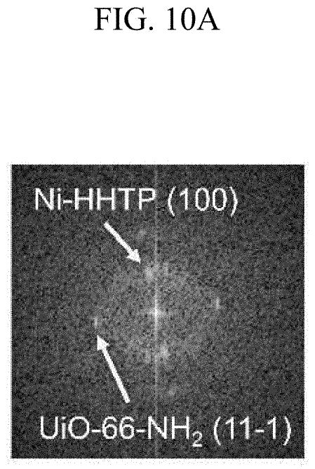

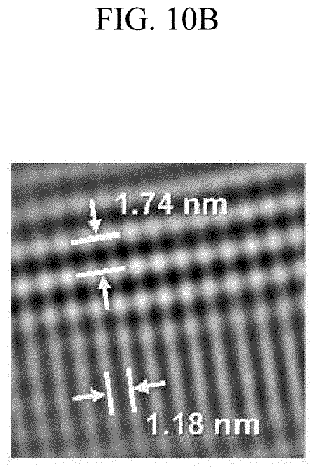

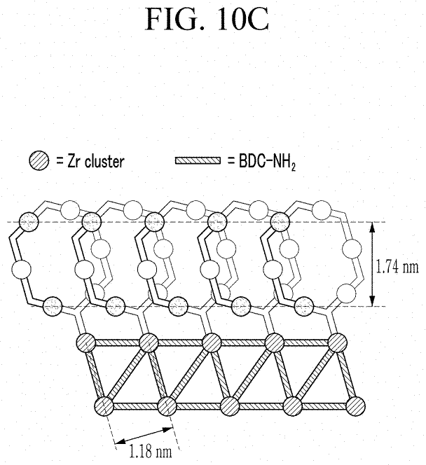

FIGS. 9, 10A, and 10B are electron microscopy images of the interface of the UiO-66-NH₂ core crystal and the Ni—HHTP rod crystals of the Ni—HHTP@UiO-66-NH₂ composites obtained in Preparation Example, and FIG. 10C is a schematic view showing microscopic image of the interface of the UiO-66-NH₂ core crystal and the Ni—HHTP rod crystal of FIG. 10B.

Referring to FIGS. 9 and 10A to 10C, at the interface of the UiO-66-NH₂ core crystal and the Ni—HHTP rod crystal of the Ni—HHTP@UiO-66-NH₂ composites obtained in Preparation Example, the lattice fringes of 1.74 nm (Ni—HHTP; the node-to-node (100) plane) and 1.18 nm (UiO-66-NH₂, (11-1) plane) are observed having seamless connectivity.

These results may indicate that the two-dimensional hexagonal layers of Ni—HHTP rod crystals are vertically aligned to the UiO-66-NH₂ core crystal, that is, the HHTP ligand is likely to form coordination bonds rather than Van der Waals interactions on the surface of the UiO-66-NH₂ core crystal.

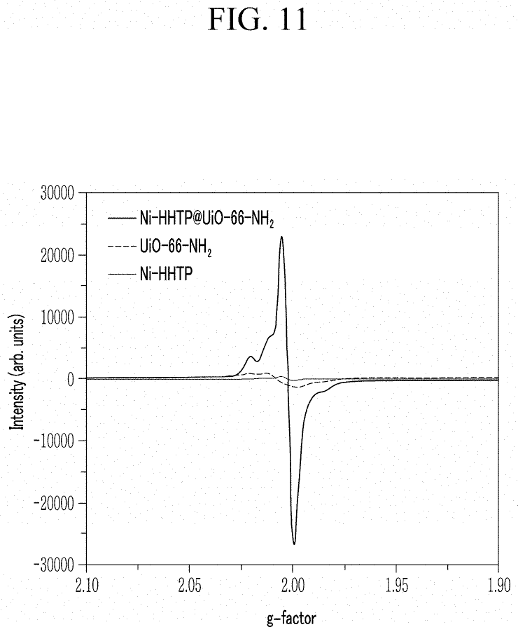

FIG. 11 is an electron paramagnetic resonance (EPR) graph of the Ni—HHTP@UiO-66-NH₂ composites obtained in Preparation Example, UiO-66-NH₂ crystals obtained in Reference Preparation Example 1, and Ni—HHTP rod crystals obtained in Reference Preparation Example 2.

Referring to FIG. 11, UiO-66-NH₂ crystals and Ni—HHTP rod crystals according to Reference Preparation Examples 1 and 2 show almost no EPR peaks, whereas the Ni—HHTP@UiO-66-NH₂ composites obtained in Preparation Example shows an intense signal at g=2.002.

From this, it may be expected that the Ni—HHTP@UiO-66-NH₂ composites obtained in Preparation Example has a semiquinone species in which the HHTP ligands are coordinated to Zr⁴⁺ instead of Ni²⁺ at the interface of the UiO-66-NH₂ core crystal and the Ni—HHTP rod crystal, and thus some of the HHTP ligands are coordinated to the metal cluster Zr⁴⁺ of the UiO-66-NH₂ core crystal.

From this, it is confirmed that Ni—HHTP@UiO-66-NH₂ composites obtained in Preparation Example are in the form of well-integrated core-shell structure by chemical bonding between the UiO-66-NH₂ core crystals and the Ni—HHTP rod crystals at the interface of the UiO-66-NH₂ core crystals and the Ni—HHTP rod crystals.

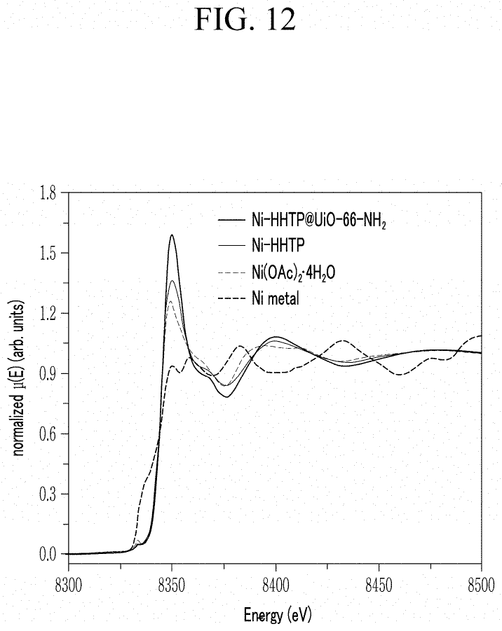

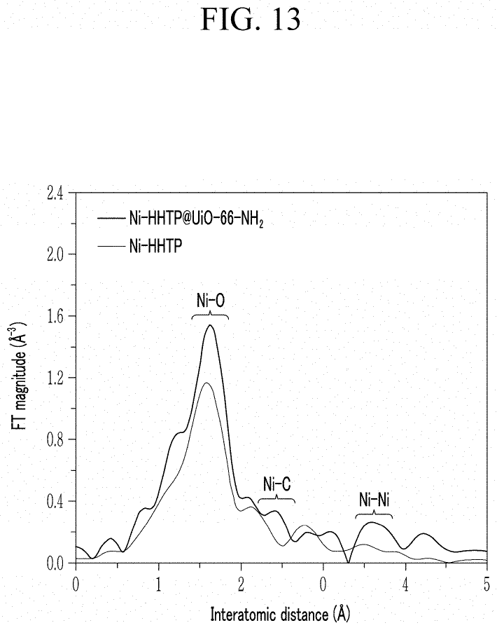

FIGS. 12 and 13 are X-ray absorption fine structure (XAFS) graphs of Ni—HHTP@UiO-66-NH₂ composites obtained in Preparation Example.

Referring to FIGS. 12 and 13, the Ni—HHTP@UiO-66-NH₂ composites obtained in Preparation Example has an intense peak different from the Ni—HHTP rod crystals obtained in Reference Preparation Example 2, and it is confirmed that Ni nodes in Ni—HHTP rod crystals adjacent to an interface of the UiO-66-NH₂ core crystal and the Ni—HHTP rod crystals of the Ni—HHTP@UiO-66-NH₂ composites obtained in Preparation Example have untypical coordination environments around the Ni nodes.

According to a more detailed analysis (k²-weighted extended XAFS), Ni—C coordination number (CN_{Ni-C}) (3.44) at the interface of the UiO-66-NH₂ core crystal and the Ni—HHTP rod crystals of the Ni—HHTP@UiO-66-NH₂ composites obtained in Preparation Example is higher than Ni—C coordination number (CN_{Ni-C}) (2.88) of the Ni—HHTP rod crystals in a region other than the interface.

Taking these results together, due to the unique interfacial properties of the UiO-66-NH₂ core crystals and the Ni—HHTP rod crystals, the Ni—HHTP@UiO-66-NH₂ composites obtained in Preparation Example may have a core-shell structure that is chemically strongly connected and homogeneously distributed, and it may effectively provide a continuous conductive pathway to be expected to exhibit improved chemical and electrical properties compared to Ni—HHTP rod crystals alone, UiO-66-NH₂ crystals alone, and/or a physical mixture (a simple physical mixture) of Ni—HHTP rod crystals and UiO-66-NH₂ crystals.

### Evaluation III

The surface areas of the Ni—HHTP@UiO-66-NH₂ composites obtained in Preparation Example and UiO-66-NH₂ crystals and Ni—HHTP rod crystals obtained in Reference Preparation Examples are evaluated.

The surface area is a BET surface area measured by Brunauer-Emmett-Teller (BET) surface area analyzer, and a reference cell not including the evaluation material and the evaluation cell including the evaluation material are supplied the same amount of N₂ gas, respectively, and then the BET surface areas are measured by the pressure difference between the reference cell and the evaluation cell due to N₂ gas adsorbed on the surface of the evaluation material.

The results are shown in Table 1.

TABLE 1

| | | BET surface |
|---|---|---|
| | | area (m²/g) |
| Preparation Example | Ni-HHTP@UiO-66-NH₂ | 1071 |
| Reference Preparation | UiO-66-NH₂ | 1320 |
| Example 1 | | |
| Reference Preparation | Ni-HHTP | 449 |
| Example 2 | | |

Referring to Table 1, porosities of the Ni—HHTP@UiO-66-NH₂ composites obtained in Preparation Example are well-maintained, despite combining the Ni—HHTP rod crystals and the UiO-66-NH₂ core crystals in a composite, and it may be expected that the Ni—HHTP rod crystals and the UiO-66-NH₂ core crystals may simultaneously provide abundant active reactive sites.

### Example: Manufacturing Chemiresistive Sensor

### Example 1

Ni—HHTP@UiO-66-NH₂ composites obtained in Preparation Example are dispersed in ethanol in 1 mg/mL to prepare a Ni—HHTP@UiO-66-NH₂ crystals dispersion.

Subsequently, the Ni—HHTP@UiO-66-NH₂ crystals dispersion is drop-coated on the glass substrate on which aluminum patterned electrodes are formed, and dried to manufacture a chemiresistive sensor.

### Reference Example 1

The chemiresistive sensor is manufactured in the same manner as Example 1, except that the Ni—HHTP rod crystals obtained in Reference Preparation Example 2 are used instead of the Ni—HHTP@UiO-66-NH₂ composites obtained in Preparation Example.

### Reference Example 2

The chemiresistive sensor is manufactured in the same manner as Example 1, except that the physical mixture of Ni—HHTP rod crystals and UiO-66-NH₂ crystals obtained in Reference Preparation Example 3 is used instead of the Ni—HHTP@UiO-66-NH₂ composites obtained in Preparation Example.

### Evaluation IV

The gas sensing performance of the chemiresistive sensors according to the Example and Reference Examples are evaluated.

The gas sensing performance is determined from the change in resistance when the chemiresistive sensor according to Example and Reference Examples are placed at room temperature (about 25° C.) and exposed to hydrogen sulfide gas (H₂S) at a concentration of 5 ppm for 60 minutes.

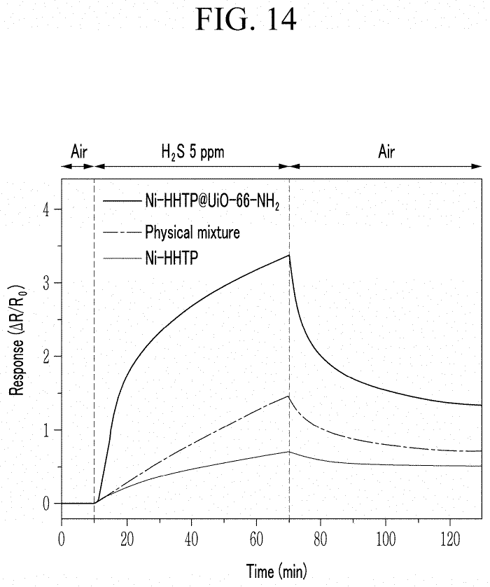

The results are shown in Table 2 and FIG. 14.

FIG. 14 is a graph showing changes in sensitivities (ΔR/R₀) when the chemiresistive sensors according to Example and Reference Examples are exposed to hydrogen sulfide gas H₂S.

TABLE 2

| | Δ R/R₀ |
|---|---|
| Example | 3.4 |
| Reference Example 1 | 0.7 |
| Reference Example 2 | 1.45 |

Referring to Table 2 and FIG. 14, the chemiresistive sensor according to Example exhibits high sensitivity for hydrogen sulfide gas (H₂S) compared to the chemiresistive sensors according to Reference Examples. Accordingly, it is confirmed that the chemiresistive sensor according to Example exhibits a synergistic effect by the above-described core-shell structured composite.

### Evaluation V

The gas sensing performance is determined when the chemiresistive sensor according to Example is placed at room temperature (about 25° C.) and exposed to hydrogen sulfide gas (H₂S) of 0.5 ppm to 10 ppm for 60 minutes.

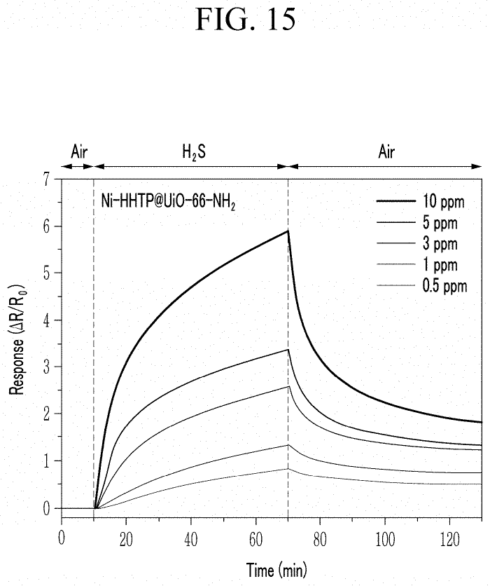

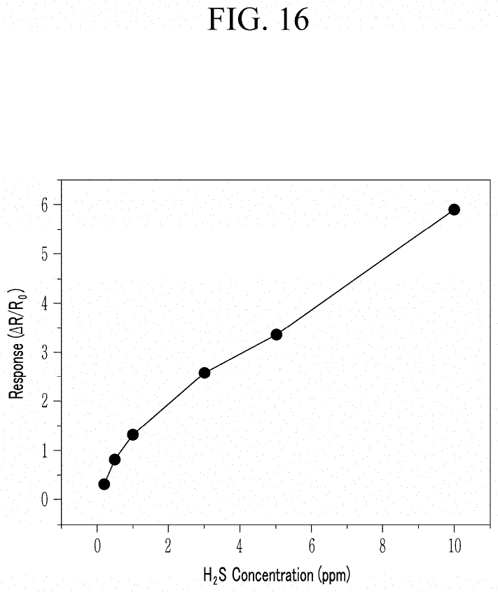

The results are shown in FIGS. 15 and 16.

FIG. 15 is a graph showing sensitivities according to gas concentration of the chemiresistive sensor according to Example, and FIG. 16 is a graph showing a relationship between gas concentration and change in sensitivities of the chemiresistive sensor according to Example.

Referring to FIGS. 15 and 16, the chemiresistive sensor according to Example exhibits changes in sensitivities in proportion to the concentration of hydrogen sulfide gas (H₂S).

### Evaluation VI

The gas selectivity of the chemiresistive sensors according to Example and Reference Examples are evaluated.

The gas selectivity is determined from changes in sensitivities when the chemiresistive sensors according to Example and Reference Examples are placed at room temperature (about 25° C.) and exposed to hydrogen sulfide (H₂S), nitrogen dioxide (NO₂), carbon monoxide (CO), ammonia (NH₃), toluene (C₆H₅CH₃), acetone (CH₃COCH₃), and ethanol (C₂H₅OH), at each concentration of 5 ppm, for 60 minutes.

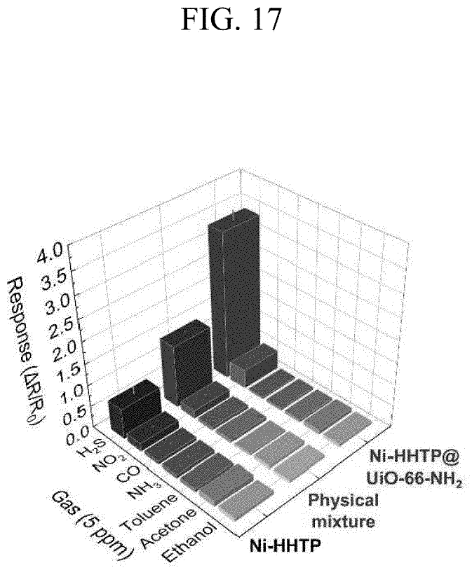

The results are shown in FIG. 17.

FIG. 17 is a graph showing gas selectivity of the chemiresistive sensors according to Example and Reference Example.

Referring to FIG. 17, it is confirmed that the chemiresistive sensor according to Example has high gas selectivity with respect to hydrogen sulfide (H₂S), and the gas selectivity with respect to hydrogen sulfide (H₂S) is higher than that of the chemiresistive sensors according to Reference Examples.

While this invention has been described in connection with what is presently considered to be practical example embodiments, it is to be understood that the invention is not limited to the disclosed embodiments. On the contrary, it is intended to cover various modifications and equivalent arrangements included within the spirit and scope of the appended claims.

What is claimed is:

1. A chemiresistor comprising
a conductive porous nanocomposite of a three-dimensional metal-organic framework and a two-dimensional metal-organic framework,
wherein the two-dimensional metal-organic framework is chemically bound to the three-dimensional metal-organic framework on a surface of the three-dimensional metal-organic framework, and
the three-dimensional metal-organic framework and the two-dimensional metal-organic framework form a core-shell structure in the conductive porous nanocomposite.

2. The chemiresistor of claim 1, wherein the two-dimensional metal-organic framework comprises a plurality of two-dimensional hexagonal layers stacked with each other, and each of the plurality of two-dimensional hexagonal layers is derived from a combination of an organic ligand having a hydrogen bonding functional group and a metal cluster.

3. The chemiresistor of claim 2, wherein the organic ligand of the two-dimensional metal-organic framework comprises a benzene ring or a fused polycyclic aromatic ring substituted with one or more hydrogen bonding functional groups selected from a hydroxy group, an amino group, a thiol group, or a combination thereof.

4. The chemiresistor of claim 2, wherein the metal cluster of the two-dimensional metal-organic framework comprises Cu²⁺, Ni²⁺, Co²⁺, Fe²⁺, Zn²⁺, Mg²⁺, or a combination thereof.

5. The chemiresistor of claim 2, wherein each of the plurality of two-dimensional hexagonal layers of the two-dimensional metal-organic framework is vertically aligned to the surface of the three-dimensional metal-organic framework.

6. The chemiresistor of claim 2, wherein
the plurality of two-dimensional hexagonal layers comprises a first two-dimensional hexagonal layer and a second two-dimensional hexagonal layer that are alternatively stacked, and
a coordination number of the metal cluster of the second two-dimensional hexagonal layer is the same as or different from a coordination number of the metal cluster of the first two-dimensional hexagonal layer.

7. The chemiresistor of claim 6, wherein the metal cluster included in the first two-dimensional hexagonal layer and the metal cluster included in the second two-dimensional hexagonal layer are arranged side by side or zigzag along a direction perpendicular to an in-plane direction of the first and second two-dimensional hexagonal layers.

8. The chemiresistor of claim 1, wherein the organic ligand of the two-dimensional metal-organic framework is coordinated with a metal cluster of the three-dimensional metal-organic framework at the interface of the three-dimensional metal-organic framework and the two-dimensional metal-organic framework.

9. The chemiresistor of claim 8, wherein the coordination number of the metal cluster of the two-dimensional metal-organic framework at the interface of the three-dimensional metal-organic framework and the two-dimensional metal-organic framework is higher than the coordination number of the metal cluster of the two-dimensional metal-organic framework in a region other than the interface of the three-dimensional metal-organic framework and the two-dimensional metal-organic framework.

10. The chemiresistor of claim 1, wherein
the three-dimensional metal-organic framework is an octahedral porous material, and
the two-dimensional metal-organic framework is a rod-shaped conductive material.

11. A method of manufacturing a chemiresistor comprising
surface-modifying a three-dimensional metal-organic framework with an organic ligand having a hydrogen bonding functional group for a two-dimensional metal-organic framework, and
providing a metal precursor for the two-dimensional metal-organic framework to the surface-modified three-dimensional metal-organic framework and then performing seed-mediated crystal growth to form a conductive porous nanocomposite of the three-dimensional metal-organic framework and the two-dimensional metal-organic framework with a core-shell structure, the two-dimensional metal-organic framework being chemically bound to the three-dimensional metal-organic framework on a surface of the three-dimensional metal-organic framework.

12. The method of claim 11, wherein the surface-modifying comprises
preparing a mixed dispersion including the three-dimensional metal-organic framework and the organic ligand for the two-dimensional metal-organic framework, and
ultrasonicating the mixed dispersion.

13. The method of claim 12, wherein the mixed dispersion further comprises N,N-diethyl form amide.

14. The method of claim 11, wherein the organic ligand for a two-dimensional metal-organic framework comprises a benzene ring or a fused polycyclic aromatic ring substituted with one or more hydrogen bonding functional groups selected from a hydroxy group, an amino group, a thiol group, or a combination thereof.

15. The method of claim 14, wherein the benzene ring or the fused polycyclic aromatic ring has six hydrogen bonding functional groups.

16. The method of claim 11, wherein the metal cluster for the two-dimensional metal-organic framework comprises a metal cation and a counter anion, and the metal cation comprises Cu²⁺, Ni²⁺, Co²⁺, Fe²⁺, Zn²⁺, Mg²⁺, or a combination thereof.

17. The method of claim 11, wherein the seed-mediated crystal growth comprises heat-treating the mixture of the surface-modified three-dimensional metal-organic framework and the metal precursor for the two-dimensional metal-organic framework at a temperature of about 40° C. to about 100° C.

18. A chemiresistive sensor comprising the chemiresistor of claim 1.

19. The chemiresistive sensor of claim 18, wherein the chemiresistive sensor is a gas sensor for detecting hydrogen sulfide.

20. A device comprising the chemiresistive sensor of claim 18.
